(19) United States
(12) Patent Application Publication
(10) Pub. No.: US 2010/0273224 A1
(43) Pub. Date: Oct. 28, 2010

(54) PRODUCTION OF LACTIC ACID BY WAY OF FERMENTATION AND EXTRACTION OF AMINES

(75) Inventors: Schulze Joachim, Soest (DE); Peter Wasserscheid, Erlangen (DE); Andreas Boesmann, Hessdorf (DE); Wolfgang Tietz, Biendorf (DE)

Correspondence Address: MARSHALL & MELHORN, LLC, FOUR SEAGATE - EIGHTH FLOOR, TOLEDO, OH, 43604, US

(73) Assignee: UHDE GMBH, Dortmund (DE)

(21) Appl. No.: 12/733,849

(22) PCT Filed: Sep. 24, 2008
(86) PCT No.: PCT/EP2008/008057

§ 371 Date: Jun. 28, 2010

(30) Foreign Application Priority Data
Sep. 24, 2007 (DE) 10 2007 045 701.6

(51) Int. Cl. C12P 7/56; C12M 1/00

(52) U.S. Cl. 435/139; 435/289.1

(57) ABSTRACT

The invention relates to a process for the production and isolation of lactic acid which is produced by way of fermentation of a carbohydrate-bearing feedstock and the addition of ammonia. The lactic acid is released from the ammonium salt of the lactate by adding a mineral acid and the lactic acid isolation takes place by extraction with the aid of an alkylated amine. The extraction is preferably operated at a pH value of 4.0 to 2.0, thereby obtaining a multi-phase mixture which is split up. The phase thus obtained with the lactate salt of amine subsequently undergoes distillation, so that lactic acid is obtained as pure product or the phase with the lactate salt of amine are thermally decomposed, thereby producing an oligolactide that is distillable and thus yields a pure dilactide. The invention also encompasses a device suitable for the performance of the inventive process.

The production of lactic acid from carbohydrate-bearing feedstock by way of fermentation is becoming increasingly important. Lactic acid is an environment-friendly product for the production of detergents, liquid soap, deliming and auxiliary substances for textiles. In recent years the interest in lactic acid has further grown because lactic acid in polymeric form, i.e. polylactide, is compostable. Polylactide or polylactic acid (PLA) is used in the form of biodegradable, biocompatible plastic materials for applications in the food and cosmetics industries and for medical appliances. Bags of compostable polylactic acid films meet with special interest because the bags of conventional plastics are not biodegradable and they are therefore considered as a major environmental impact. Plastic bags based on polylactic acid, however, are biodegradable and thus regarded as an environmental alternative to bags of conventional plastic materials.

The feedstock for lactic acid production is a carbohydrate-bearing material converted to lactic acid by appropriate micro-organisms. Suitable bacteria for this purpose are, for example, lactic acid bacteria from the lactobacillaceae strain, but also micro-organisms from the saccharomyces or rhizopus strains. Depending on the strain of micro-organisms used, either the laevorotatory enantiomer or dextrorotatory enantiomer of the lactic acid are obtained. Thus, for example, the application of the lactobacillus bulgaris exclusively yields the laevorotatory enantiomer, d-(−) lactic acid. When using a different strain, i.e. the lactobacillus casei strain, you exclusively obtain the other enantiomeric form, i.e. l-(+) lactic acid.

During the fermentative conversion of carbohydrates to lactic acid, the pH value of the fermentation broth decreases due to the formation of the acid. At a high concentration of lactic acid, the pH value may drop to values as low as 2.0. If the acid exhibits an equimolar concentration with the salt, the pH value of the liquor is 3.86, which equals the pKₐ value of the lactic acid (at 25° C.). Several strains of bacteria can produce lactic acid at a lower pH value but the majority of strains require a higher pH value for the production of lactic acid. For this reason it is common practice to add a basic salt to the fermentation broth in the lactic acid production so that the pH value rises to 5.0 or higher. The said basic salt often is a basic calcium salt that forms sparingly soluble calcium salts with the lactic acid produced.

The calcium salt obtained can be easily separated from the fermentation process because it is sparingly soluble. For release of the lactic acid, the isolated calcium salt is slightly acidified with the aid of a mineral acid such that the lactic acid is released and the calcium salt of the mineral acid is obtained. When sulphuric acid is used as mineral acid, sparingly soluble calcium sulphate is formed and can be easily removed from the process by filtration.

It is also possible to remove the lactic acid from the lactic acid solution by way of extraction. Although lactic acid is very hydrophilic and cannot be readily separated from an aqueous solution, there are various solvents that are suitable for the extraction of lactic acid from an aqueous solution. Solvents frequently used for this purpose are trialkylphosphates or trialkylphosphine oxide which form a two-phase system with water and can easily dissolve the lactic acid on account of a sufficiently high distribution coefficient. Solvents often used, too, are tri-n-alkylamines which extract lactic acid from water.

It is likewise possible to use an amine for neutralisation in a fermentation process instead of a calcium salt. In this case it is crucial to ensure compatibility of the microbial strain with the respective amine. Patent specification WO 2006/124633 A1 describes a process for the production of ammonium lactate by way of fermentation. During fermentation the ammonium salt of the lactic acid forms and can be separated from the fermentation broth by extraction, for example. In a downstream step, the ammonium salt can be split up very easily with the aid of a weak acid or carbon dioxide. This method yields free lactic acid which, for example, can be purified by distillation.

The direct extraction of hydroxycarboxylic acid from fermentation processes was investigated, too. This method will work properly if a solvent is used which is non-toxic to the strains of bacteria producing the lactic acid and if the pH value of the fermentation broth is exactly adjusted to the pH value specified. Hano et al. reported in a 1993 publication Bioseparation 3, pages 321-326, on an extraction that was carried out at a pH value of pH=5, di-n-octylamine being used as solvent. When that method is performed after the fermentation at a pH value of pH=2 to 2.5, it is possible to extract the lactic acid with the aid of a combined solvent of di-n-octylamine and hexane. In order to remove the solvent from the lactic acid, the solvent is treated with ammonia prior to the distillative separation step.

These processes have the advantage of an easy handling compared to the conventional production methods of lactic acid with the aid of calcium lactate. As no sparingly soluble calcium salt is obtained, which must be separated from the solution by a sophisticated filtration method and be disposed of, the scope of equipment is by far smaller, on condition that optimum pH values are found for the extraction and fermentation and that appropriate solvents are available which are non-toxic to the strain of bacteria producing the lactic acid, form a two-phase system with water and have a sufficiently high potential of extraction for lactic acid.

Although it is easier from the technical point of view to perform an extractive distillation instead of carrying out an isolation with the aid of calcium salt, this method has the disadvantage that an adequate solvent combination is not always readily available for the above-mentioned reasons. Furthermore, it may happen that the solvent co-extracts a part of the carbohydrates, too, which leads to a poorer yield of the whole process and thus makes the product isolation more difficult. Moreover, several extraction cycles are often required to completely separate the lactic acid from the aqueous fermentation agent. Depending on the type of fermentation process, the solvent mixtures needed for the extraction necessitate a sophisticated method for removal from the aqueous phase depending on the phase formation.

Therefore, the objective of the invention is to provide a combined production and extraction process for lactic acid originating from a fermentation reactor. During the extraction the pH value of the fermentation broth must preferably be lower than the pKₛ value of the lactic acid. The separation and treatment of the extract must be unsophisticated from the process point of view and supply lactic acid of high purity at a high yield rate. The solvent to be used must be free from additional components and form a two-phase mixture with water. In addition, the lactic acid production according to the invention must permit an easy integration of a process step that also allows the production of oligolactide or polylactide.

The objective of the invention is achieved by an extraction process using a linear n-trioctylamine (TOA) or another suitable amine as solvent. n-trioctylamine covers a miscibility gap with water in the phase diagram such that it is easy to separate the said solvent by means of a phase-separation device, provided a given mixing ratio is observed. The lactate formed from lactic acid is also liquid, covers a miscibility gap with water in the phase diagram and is likewise easily separable from the water and the input amine. The lactate can be readily split up with the aid of heat so that it is in fact easy to obtain lactic acid when a distillation of the formed ammonium lactate is carried out. No additional solvents are needed to adjust the correct solvent properties.

The objective of the invention is achieved, in particular, by a process for the production of lactic acid from carbohydrate-bearing feedstock, thereby performing at least one fermentative process step, the said process featuring the following technical details:
A carbohydrate-bearing feedstock is converted in a first process step of the fermentation in a fermentation reactor to form an ammonium lactate-bearing solution in the presence of micro-organisms and ammonia, and
the ammonium lactate-bearing solution thus obtained undergoes extraction in the next process step with the aid of a mineral acid and alkylated amine, and
the mixture thus produced is thoroughly mixed or stirred, thereby obtaining by extraction a three-phase mixture, the first phase mainly consisting of the alkylated amine, the second phase mainly of the salt of alkylated amine and lactic acid, and the third phase mainly of water and ammonium sulphate, and
the three-phase mixture thus obtained is split up into three phases in a device for phase separation, and
the second phase obtained, which primarily consists of the salt of alkylated amine and lactic acid, undergoes distillation, which yields lactic acid, alkylated amine and a high-boiling distillation residue, and
the biological fermentation residues obtained by the fermentation are removed from the system either directly after the fermentation, after the extraction, during phase separation or during distillation.

The following feedstocks are particularly suitable as carbohydrate-bearing feedstocks for fermentation: starch-bearing materials such as rice and potato starch, pulp residues, hydrolysates and corn starch. But sugar-bearing materials are likewise suitable, such as dextrose, saccharose, glucose and hexose, in general. It is also possible to make use of carbohydrate-bearing materials originating from wood, such as xylan, xylose and pentose, in general. It goes without saying that mixtures of carbohydrates are likewise suitable as feedstock for the fermentative production of lactic acid.

The micro-organisms suited for producing lactic acid from carbohydrates are, in particular, the lactobacillaceae strains. Micro-organisms particularly suited for performing the process specified in this invention originate from the lactobacillus casei genus. Bacterial strains of the rhizopus, pediococcus or saccharomyces genus are likewise suitable for the inventive process. Moreover, any micro-organism capable of converting carbohydrates to lactic acid or lactates are also suited for the said process. If strains of the lactobacillus or pediococcus genus are employed, the fermentation can take place at moderate pH values of 5.0 to 7.0. But the micro-organisms preferred for the inventive process are those that produce lactic acid at pH values of ≦3.8. These specific micro-organisms originate from the lactobacillus genus described, for example, in EP 1025254 A1.

The fermentation itself takes place, for example, batchwise in a nutritive solution at a temperature of 20 to 60° C. and a pH value of 4.0 to 2.0. The preferred temperature for fermentation is 25 to 35° C. and the preferred pH value is 3.0 to 2.5. In order to adjust the optimal pH value, the fermentation broth is mixed with a mineral acid if need be, preferably sulphuric acid or phosphoric acid. Furthermore, an ammonia solution is added to the fermentation broth to achieve an optimum pH value. Depending on the formation of lactic acid, it is also possible to carry out post-dosing to keep the pH value constant.

After fermentation, the fermentation broth is sent to an extraction vessel and mixed with n-trioctylamine. For lactic acid release, mineral acid is added to the ammonium lactate. Upon adding n-trioctylamine the mixture is thoroughly stirred or shaken so that a three-phase mixture is obtained. The first phase mainly consists of pure tri-n-octylamine, the second phase mainly of tri-n-octylammonium lactate and the third phase essentially of an aqueous ammonium sulphate solution. The pH value should range from 4.0 to 2.0 to obtain an optimal phase separation, but it may also exceed this value. As a rule, the temperature is not changed for the extraction and may range from 25 to 35° C.

The three-phase mixture thus obtained is subsequently sent to a device for phase separation in which three phases are produced: the surplus tri-n-octylamine, tri-n-octylammonium lactate and the aqueous phase. According to a beneficial embodiment of the invention, the tri-n-octylamine is recycled to the process via adequate piping and devices. The aqueous phase is disposed of and it still contains ammonium sulphate and max. 2% by weight of lactic acid. The latter can be separated by adequate methods, for example, by a diffusion membrane. Tri-n-octylammonium lactate undergoes further processing.

In order to facilitate the reaction parameters it is possible to separate the solid fermentation residues obtained by fermentation and essentially consisting of cell residues and solid metabolites, using an appropriate device which in this case can be done by advantageous filtration equipment. But other separation devices are also suitable, provided they are well suited for the removal of solid fermentation residues. According to a beneficial embodiment of the invention, the said separation device for solid fermentation residues is arranged directly downstream of the fermentation reactor. However, it is also possible to integrate the said separation device downstream of the extraction unit or downstream of the device for the treatment of lactate. The fermentation residues thus obtained are normally solids and useable for further applications or they must be disposed of.

It is feasible to improve the quality of the produced lactic acid by further process steps, such as the addition of chemicals for decolorisation. This step preferably takes place after fermentation, but it can likewise be performed at any stage of the process.

According to an embodiment of the invention, the tri-n-octylammonium lactate originating from the phase separation undergoes further processing, which can be carried out by means of a distillation device. Owing to the boiling point of the lactic acid (122° C., 20 hPa) the lactic acid is preferably distilled under vacuum. Tri-n-octylammonium lactate is rapidly thermolysed so that tri-n-octylamine and lactic acid are obtained. It is easy to produce pure lactic acid by this method. In fact, a further product obtained is a sparingly soluble and high-boiling bottom substance that mainly consists of tri-n-octylammonium sulphate and oligolactates.

In accordance with a further embodiment of the invention, the tri-n-octylammonium lactate originating from the phase separation and the surplus tri-n-octylamine are heated for further processing. In order to perform this partial step according to the invention, temperatures of 250° C. to 350° C. are required, thereby obtaining essentially tri-n-octylamine and a liquid oligolactide that can be further distilled. These temperatures are required to ensure a quick adjustment of the equilibrium with the oligolactide. It is recommended that the thermolysis be carried out at 300° C. in order to ensure that the oligolactide forms in a sufficiently short period to prevent the decomposition of the substances.

An evaporator is also suited for the thermolysis and for this purpose the tri-n-octylamine lactate is sent through the evaporator filled with packings, which facilitates a decomposition of the lactate. Such packings preferably consist of acidic oxides. It is also possible to use other types of packing. γ-aluminium oxide, for example, is well suited for this purpose. In principle, any packing that permits a split of the tri-n-octylamine lactate to form amine and lactic acid and subsequently oligolactide can be used for this step.

In order to increase the temperature for the thermolysis, an inert gas can be fed to the evaporator, i.e. any gas that does not react with lactate is suitable. A preferred inert gas is dry argon but it is also possible to use cheaper nitrogen. The tri-n-octylamine obtained can be recycled to the process. The subsequent distillation of oligolactide under high vacuum yields pure dilactide and a sparingly soluble bottom substance that mainly consists of tri-n-octylammonium sulphate. Pure dilactide can be isolated.

The extraction amine is recovered during the phase separation and the downstream distillation or thermolysis and is recycled to the process for cutting the costs. The amine originating from the extraction and the recovered amine can be united to form one stream or be returned to the process via different routes. Depending on the amine purity, the amine can also be subjected to a purification step prior to recycling it to the process. The purification steps suited for this purpose are a renewed distillation, filtration or a purification by the membrane method.

For the lactic acid extraction, any amine can be used for the inventive process provided the amine has a sufficient solubility for the lactic acid, exhibits a miscibility gap with water and forms a lactate that also exhibits a miscibility gap with water. The alkylated amine should have a water solubility which preferably amounts to <1% by mass at 25° C. The amine used for the extraction, however, preferably possesses a water solubility of <0.1% by mass. The lactic acid salt of the amine should have a watersolubility which likewise preferably amounts to <1% by mass. The ammonium lactate obtained preferably has a watersolutibility of <0.1% by mass and, as a rule, it is liquid. Hence, it is pumpable and preferably conveyed in liquid state by pumps.

The amines suited for the process are those which are of a primary, secondary or tertiary nature. The substituents of the amine are preferably hydrocarbon residues, which in this case are understood to be randomly constituted substituents such as residues of alkyl, iso-alkyl, cycloalkyl, aryl or substituents which themselves are substituted by one of the said substituents, such as arylalkyl substituents. The preferred form of the alkylated amine is such that it exhibits an overall C-number of 10 carbon atomes in the substituents. In fact, it is also feasible to use hydrocarbon residues linked with foreign substituents, such as halogen substituents or nitrile substituents. However, tri-n-octylamine is preferably used for the inventive process.

Depending on the bacterial strain used, the inventive process also allows to produce pure l-(+) enantiomer or pure d-(−) enantiomer. Depending on the enantiomer obtained and optical purity of the produced mixture of enantiomers, the lactic acid product formed has different physical properties. Thus, an enantiomerically pure lactic acid has a melting point of 53° C., but a racemate a melting point of 16.8° C.

The inventive process can be performed with a device suited for compliance with the requirements involved. The claim encompasses, in particular, a device featuring the following technical criteria:
The device includes a reactor suitable for fermentation processes, and
an extraction vessel is arranged downstream of the fermentation reactor, and
a device for phase separation is installed downstream of the extraction vessel, and
a distillation column is integrated downstream of the device for phase separation.

In order to carry out the further process steps involved, it is recommended that residues originating from the fermentation process be removed. Therefore, an embodiment of the invention provides for a device which is capable of removing the fermentation residues and integrated downstream of the fermentation reactor. The said items of equipment can operate batchwise or in continuous operation.

A cross-flow filter is a well suited unit for this task. This filter permits a continuous and efficient clarification of the fermentation broth. But it is also feasible to use a screening unit or a centrifuge for removing the fermentation residues. Typical centrifuges for the removal of fermentation residues are decanter or tubular bowl centrifuges. Depending on the design of the inventive device, the unit for removing the fermentation residues may also be installed further downstream in the process itself. This applies, for example, in case the removal of the fermentation residues is envisaged at the level of the tri-n-octylammonium lactate distillation.

In practice it is also possible to carry out the extraction in a membrane extractor. A prerequisite for such an operation is to provide a membrane which is permeable to lactic acid. Suitable types of membrane are, for example, made from polyether sulphones or polytetrafluoroethene. In this case, the fermentation residues remain on the fermentation side of the membrane. The side of the membrane permeable to lactic acid is wetted with amine such that the corresponding ammonium lactate can form. It is recommended that the phase separation equipment be installed downstream of the membrane reactor which is used to separate the amine from the ammonium lactate, the amine being returned to membrane reactor.

For extraction it is possible to use vessels that permit a rapid mixing of the fermentation broth with amine. The said vessels are equipped with devices for an efficient and thorough stirring or shaking of the vessel. Additionally they can be provided with metering/dosing devices which allow a precise addition of amine and mineral acid to initiate neutralisation. As a rule, the extraction is carried out batchwise but it may also take place in continuous operation. Upon ending the extraction, a mixture with three phases is obtained in discontinuous manner and it consists of ammonium lactate, amine and the ammonium sulphate-bearing aqueous phase.

Devices suitable for phase separation are installed downstream of the extraction unit. Here the aqueous phase is separated from the amine phase and ammonium lactate phase. The aqueous phase is removed by appropriate devices and disposed of. Devices suitable for phase separation are, for example, decanters or gravity separators.

According to an embodiment of the invention, the amine and ammonium lactate-bearing phases then undergo distillation. Devices suited for distillation are those vessels which can distillate phase mixtures of ammonium lactate/amine. Since the lactic acid undergoes vacuum distillation because of its boiling point (122° C., 20 hPa), the distillation device has gadgets required to maintain the vacuum. During heating of the phase mixture of alkylammonium lactate/amine, the alkylammonium lactate decomposes such that essentially amine and lactic acid distil over. In order to improve the separation effect, the distillation device can be equipped with a column head, bubble trays or other gadgets that facilitate the separation effect. The products obtained are lactic acid and alkylamine. The bottom mixture that cannot be distilled essentially consists of alkylammonium sulphate and oligolactates and is treated and disposed of by adequate equipment items.

According to a further embodiment of the invention, the amine and ammonium lactate-bearing phase undergoes a thermolysis upon separating the aqueous phase. For this purpose any vessel permitting heating and efficient mixing of the content is well suited. In accordance with a beneficial embodiment of the invention, it is also feasible to use an evaporator such as specified in patent specification WO 92/05168 A1. A beneficial embodiment of the invention provides for a packing of the evaporator, the said packing consisting of acidic oxide. Typical acidic oxides are γ-aluminium oxides, but silica, silica/aluminium oxide combinations or zeolites are suitable, too.

The said evaporator may also have a device appropriate for the passage of inert gas that is propellant for the amine passing through the evaporator. In order to separate the lactic acid from the amine, a distillation column is installed downstream of the evaporator and suited for removing the lactic acid from the amine by distillation. The device that may be arranged upstream of the distillation column is capable of the phase separation of the content.

In order to recycle the amine originating from the extraction, thermolysis and distillation into the process, the said devices are equipped with gadgets which allow a return of the amine into the process. As the lactate of the used amine is pumpable, the scope of equipment also includes the necessary piping, pumps and valves. It is also possible to add items of equipment permitting a purification of the amine. A typical example is a distillation column which may be equipped with items improving the separation effect, such as bubble trays or packed columns. It is also feasible to provide filtration units for the purification of the amine or any other device suited for amine purification. If need be, one may also provide an instrument upstream of the said device in order to measure the amine purity, such as an instrument for the measurement of the index of refraction.

When the dilactide originating from the thermolysis undergoes distillation you obtain product dilactide, which is subsequently sent to devices suited for further processing. The lactic acid produced or the dilactide may be used for any purpose desired. It is possible to use the lactic acid for environment-friendly detergents, deliming agents or cosmetical substances. It is likewise possible to use the lactic acid for the conversion of chemicals or polylactic acid, the latter being a good multi-purpose and workable plastic material with good biodegradation properties. Moreover, polylactic acid is suitable for the production of appliances for daily life, for medical appliances or implantation tasks or even as packing material. Polylactic acid is in particular well suited for the production of plastic bags that are biodegradable. EP 1247808 A2 document describes a typical process for the production of polylactides.

The process described is suited not only for the production of lactic acid but also generally for the commercial-scale production of α-hydroxycarboxylic acid. The inventive process is well suited for a cost-efficient availability of large quantities of lactic acid. The said process does not require the addition of calcium salt nor a subsequent acidification so that no sparingly soluble calcium salts must be disposed of. The extraction necessitates no sophisticated processing and permits a simple purification of the lactic acid. The extraction agent used is non-toxic to micro-organisms and is applicable at neutral to acidic pH values, but preferably at acidic pH values. Depending on the microbial strain used, a high yield of lactic acid is obtained. The lactic acid thus produced is of high purity and quality.

The rate of amine recovery amounts to >90% so that low costs are incurred for the amine input during operation. Amine does not dissolve carbohydrates, which avoids losses in feedstock during the extraction.

The invention in particular contains a Claim covering the production of lactic acid isolated in line with the inventive process. If using an adequate strain of bacteria it is also possible to produce lactic acid by the inventive process, the lactic acid obtained being enantiomerically pure. This Claim also encompasses the production of enantiomerically pure or enantiomerically enriched lactic acid, the said acid being isolated by the inventive process.

The invention also includes a Claim relating to the production of oligolactides and polylactides derived from the lactic acid produced in line with the inventive process. If only one of the lactic acid enantiomers produced by the inventive process is used, the production of oligolactides or polylactides also yields the said products which possess the corresponding stereo-chemical configurations. The present invention likewise encompasses a Claim for the production of polymers derived from enantiomerically pure or enantiomerically enriched lactic acid. The invention also includes a Claim for the production of PLA co-polymers derived from lactic acid produced in line with the inventive process.

The embodiment of the invention relating to the production of lactic acid is illustrated on the basis of two flowsheets attached; it is emphasised that the inventive process is not restricted to the configurations shown in the said drawings.

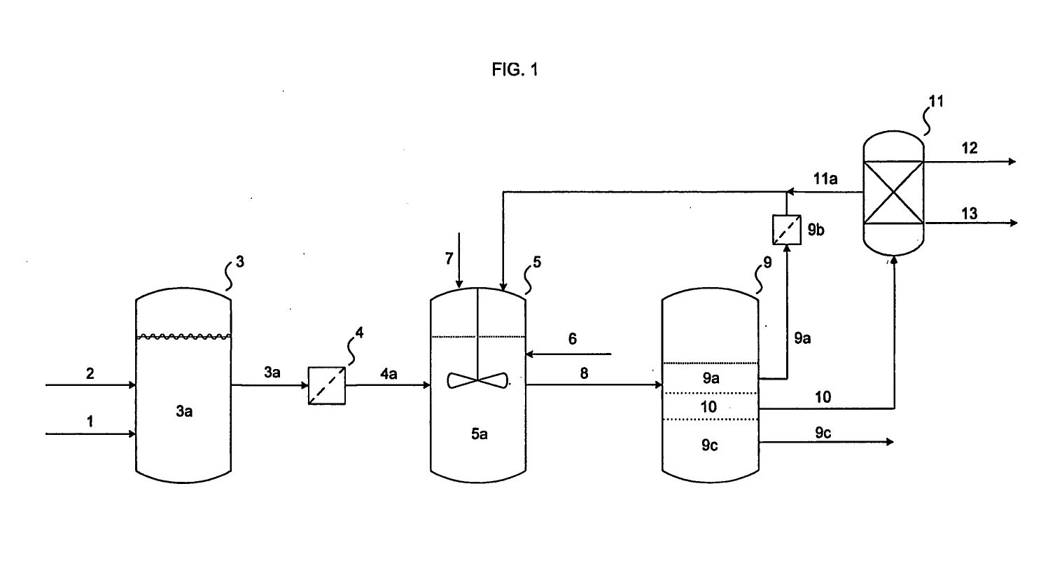

FIG. 1 shows an inventive device for the performance of the process described in this invention, the lactic acid being produced by direct distillation of the ammonium lactate. A carbohydrate-bearing aqueous solution (1) is fed to a fermentation vessel (3), the pH value being increased by the addition of ammonia (2), depending on the fermentation progress. The ammonium lactate-bearing solution (3a) thus obtained is sent to an extraction vessel (5) after the end of the fermentation process. According to the configuration described here, the fermentation broth is piped to a device (4) well suited for the removal of solid fermentation residues so that the extraction vessel is fed with a solution already clarified (4a). The said extraction vessel is equipped with feeding devices which permit the addition of mineral acid (6) to the fermentation broth for initiating the neutralisation. The amine (7) is likewise added to the extraction unit via adequate feeding devices. The extraction solution (5a) is subsequently stirred or shaken for extraction. Thus, a mixture (8) of three phases is obtained, the mixture consisting of amine, ammonium lactate and an aqueous phase. The mixture is sent to a vessel (9) that is capable of phase separation. The aqueous, ammonium sulphate-bearing phase (9c), which still contains traces of amine sulphate salt, is withdrawn from the unit. The amine-bearing phase (9a) is likewise separated and if need be, undergoes purification in a purification device (9b) and it is subsequently recycled to the extraction unit. The third phase (10), which essentially consists of the lactate of amine and minor portions of amine and sulphate salt of the lactate, undergoes distillation in high vacuum. During the distillation (11) the salt of amine and of lactic acid decomposes so that pure lactic acid (12) is obtained as product, which distills over at a temperature of 122° C. (20 hPa). The amine originating from the distillation (11a) is recycled to the extraction unit. A sparingly soluble distillation bottom product (13) is likewise obtained and mainly consists of the amine sulphate salt. The lactic acid (12) subsequently undergoes further processing.

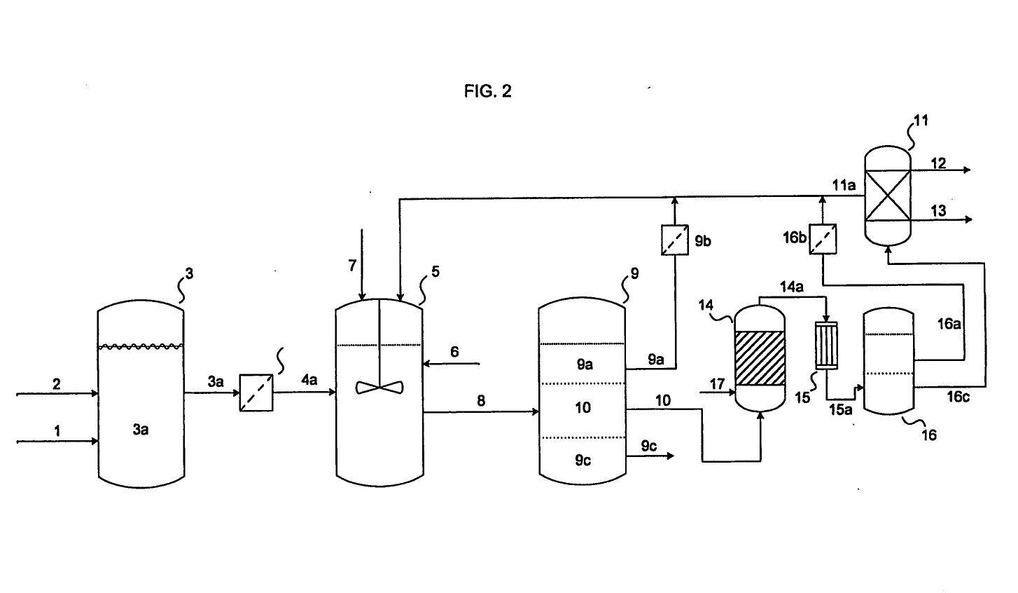

FIG. 2 shows an inventive device for the performance of the process described in the present invention, dilactide being produced by thermolysis of the oligolactide. A carbohydrate-bearing, aqueous solution (1) is fed to the fermentation vessel (3), the pH value being increased by the addition of ammonia (2), depending on the fermentation progress. The ammonium lactate-bearing solution (3a) thus obtained is processed after fermentation by a device suitable for purification (4) and consequently sent to the extraction vessel (5) in a clarified state. The said extraction vessel is equipped with feeding devices which permit the addition of mineral acid to the fermentation broth for initiating neutralisation. The amine (7) is likewise added to the extraction solution via adequate feeding devices. The said solution is subsequently stirred or shaken for extraction. Thus, a mixture (8) of three phases is obtained, the mixture consisting of amine, lactate of amine and an aqueous phase. The mixture is sent to a vessel (9) that is capable of phase separation. The aqueous, ammonium sulphate-bearing phase (9c), which still contains minor traces of amine sulphate salt, is withdrawn from the unit. The amine-bearing phase (9a) is likewise separated and if need be, undergoes purification in the device (9b) and it is subsequently recycled to the extraction unit. The lactate-bearing phase (10), which essentially consists of the lactate salt of amine, is evaporated and sent to the evaporator (14) loaded with oxidic packings. In order to facilitate the evaporation, inert gas (17) can be added to the evaporating lactate stream, in which the lactate salt of amine is evaporated and decomposed. The evaporation step and thermolysis (14) yield the gaseous amine and gaseous oligolactate as product (14a). This mixture is condensed in the condenser (15) and the condensate (15a) is piped to the phase separator (16) in which the amine (16a) is recovered. In case of need, the recovered amine is purified in an adequate device (16b) and is returned with the feed stream to the extraction device to undergo extraction again. The oligolactate (16c) undergoes high-vacuum distillation (11), which yields further amine (11a) and pure dilactide (12). A high-boiling distillation bottom product (13) is likewise obtained and mainly consists of amine sulphate salt. The additional amine (11a) can be recycled to the process.

### KEY TO REFERENCED ITEMS

1 Carbohydrate solution (in water)
2 Aqueous ammonia solution
3 Fermentation vessel
3a Fermentation broth
4 Purification device
4a Clarified fermentation broth
5 Extraction vessel
6 Mineral acid
7 Alkylated amine
8 Extraction liquor
9 Phase separator
9a Phase with alkylated amine
9b Purification device
9c Aqueous phase
10 Phase with lactate salt of alkylated amine
11 Distillation unit
11a Alkylated amine
12 Lactic acid
13 High-boiling distillation bottom product
14 Evaporator
14a Thermolysate (alkylated amine and oligolactide)
15 Condenser
15a Condensate
16 Phase separator
16a Phase with alkylated amine
16b Purification device
16c Oligolactide
17 Inert gas

1-39. (canceled)

40. A Process for the production of lactic acid from carbohydrate-bearing feedstock, the process comprising at least one fermentative process step, wherein:
a carbohydrate-bearing feedstock is converted in a first process step of the fermentation in a fermentation reactor to form an ammonium lactate-bearing solution in the presence of micro-organisms and ammonia, and
the ammonium lactate-bearing solution thus obtained undergoes extraction in the next process step with the aid of a mineral acid and alkylated amine, and
the mixture thus produced is thoroughly mixed or stirred, thereby obtaining by extraction a three-phase mixture, the first phase mainly comprising the alkylated amine, the second phase mainly the salt of alkylated amine and lactic acid, and the third phase mainly water and ammonium sulphate, and
the three-phase mixture thus obtained is split up into three phases in a device for phase separation, and
the second phase obtained, which primarily consists of the salt of alkylated amine and lactic acid, undergoes distillation, which yields lactic acid, alkylated amine and a high-boiling distillation residue, and
the biological fermentation residues obtained by the fermentation are removed from the system either directly after the fermentation, after the extraction, during phase separation or during distillation.

41. The process in accordance with claim 40, wherein the amine obtained in phase separation is recycled to the extraction process.

42. The process in accordance with claim 40, wherein the alkylated amine originating from the distillation arranged downstream of the extraction is recycled to the extraction process.

43. The process in accordance with claim 40, wherein the alkylated amine comprises an alylated amine with a water solubility of <1% by mass at 25° C. and whose lactic acid salts likewise have a water solubility of <1% by mass.

44. The process in accordance with claim 40, wherein the alkylated amine is of a primary, secondary or tertiary nature.

45. The process in accordance with claim 40, wherein the respective alkylated amine belongs to those that exhibit an overall C-number of 10 carbon atoms in the substituents.

46. The process in accordance with claim 40, wherein the substituents of the alkylated amine are alkyl, iso-alkyl, cycloalkyl, aryl or arylalkyl substituents.

47. The process in accordance with any claim 40, wherein the alkylated amine is trioctylamine.

48. The process in accordance with claim 40, wherein the mineral acid required for acidification in the extraction is sulphuric acid.

49. The process in accordance with claim 40, wherein the mineral acid required for acidification in the extraction is phosphoric acid.

50. The process in accordance with claim 40, wherein the pH value upstream of the extraction unit is below the pKₛ value of the lactic acid.

51. The process in accordance with claim 40, wherein the pH value upstream of the extraction is decreased to a value of <3.

52. The process in accordance with claim 50, comprising heating the lactate-bearing phase obtained after the extraction, thereby producing an oligolactide and the alkylated amine by way of thermolysis and oligomerisation.

53. The process in accordance with claim 52, wherein the thermolysis and oligomerisation take place at a temperature of 250° C. to 350° C.

54. The process in accordance with claim 52, wherein the lactate-bearing phase obtained after the extraction is penetrated by an inert gas while being heated for the production of oligolactide.

55. The process in accordance with claim 54, wherein the inert gas comprises argon.

56. The process in accordance with claim 54, wherein the inert gas comprises nitrogen.

57. The process in accordance with claim 52, comprising recycling the alkylated amine obtained by way of thermolysis to the process of extraction.

58. The process in accordance with claim 40, wherein the production of lactic acid yields stereoselectively the l-(+) enantiomer and the lactides derived therefrom possess the resulting configurations.

59. The process in accordance with claim 40, wherein the production of lactic acid yields stereoselectively the d-(−) enantiomer and the lactides derived therefrom possess the resulting configurations.

60. The process in accordance with claim 52, comprising subsequent distillation of the oligolactide obtained by way of thermolysis and oligomerisation, thereby producing pure dilactide.

61. The process in accordance with claim 40, wherein the process step of extraction of the lactate-bearing solution is carried out in a membrane reactor equipped with a membrane permeable to lactic acid.

62. The process in accordance with claim 40, wherein the lactic acid produced is converted to lactide directly after distillation in the vaporous phase with the aid of an adequate catalyst.

63. The process in accordance with claim 40, wherein the feedstock for the fermentative lactic acid production is saccharose.

64. The process in accordance with claim 40, wherein the feedstock for the fermentative lactic acid production is a mixture of hexoses.

65. The process in accordance with claim 40, wherein the feedstock for the fermentative lactic acid production are hexoses or pentoses or a mixture of these carbohydrates.

66. The process in accordance with claim 40, wherein suitable strains of bacteria are used as micro-organisms for the lactic acid production.

67. The process in accordance with claim 40, wherein strains of bacteria originating from the lactobacillaceae genus are used as micro-organisms for the lactic acid production.

68. The process in accordance with claim 40, wherein the fermentation broth contains nitrogen-bearing nutritive materials.

69. The process in accordance with claim 40, wherein the fermentation takes place at a temperature of 20° C. to 60° C.

70. The process in accordance with claim 40, wherein the product originating from the fermentation is treated with adequate chemicals for decolorization.

71. A device for carrying out a process in accordance with claim 40, comprising:
a reactor suitable for fermentation processes,
an extraction vessel arranged downstream of the fermentation reactor,
a device for phase separation installed downstream of the extraction vessel, and
a distillation column integrated downstream of the device for phase separation.

72. The device in accordance with claim 71, comprising a device for the removal of biological fermentation residues integrated between the fermentation reactor and the extraction vessel.

73. The device in accordance with claim 72, wherein the device for the removal of biological fermentation residues is a precoat filtration, an ultrafiltration or a simulated moving-bed filtration unit.

74. The device in accordance with claim 73, wherein the extraction vessel has a liquid inventory which can be stirred.

75. The device in accordance with claim 71, comprising a device for thermolysis and oligomerisation which is tied into the process between the phase separation unit and the distillation column.

76. The process in accordance with claim 75, wherein the distillation device is equipped with gadgets for maintaining the vacuum.

77. The device in accordance with claim 76, wherein the device for thermolysis and oligomerisation encompasses an evaporator filled with a packing.

78. The device in accordance with claim 77, wherein the packing comprises γ-aluminium oxide.
